(19) United States
(12) Patent Application Publication
(10) Pub. No.: US 2015/0173651 A1
(43) Pub. Date: Jun. 25, 2015

(54) COLPOMETER - DEVICE FOR MEASURING THE DIAMETER OF THE VAGINAL FORNIX

(71) Applicant: Arthur BERGER EINAR, (US)

(72) Inventor: Einar Arthur Berger, Sao Leopoldo (BR)

(21) Appl. No.: 14/404,729

(22) PCT Filed: May 10, 2013
(86) PCT No.: PCT/BR2013/000161

§ 371 Date: Dec. 1, 2014

(30) Foreign Application Priority Data
Jun. 1, 2012 (BR) BR 1020120132613

(51) Int. Cl. A61B 5/107

(52) U.S. Cl. CPC A61B 5/1076

(57) ABSTRACT

Colpometer—device for measuring the diameter of the fundus vaginalis is an instrument for medical use that does not exist on the market and is produced from AISI 420 stainless steel for reuse after autoclave sterilization or produced from polyethylene polymer for disposable use. The device allows measurement of the diameter of the vaginal fornix to enable the medical professional to ascertain said measurement and thus to selected the size of intravaginal component to be used. It has a curved rod with a graduated scale in centimeters that is capable of measuring the distance between the distal ends of the instrument when open. The distance between the open distal ends inside the fundus vaginalis allows verification, using the centimeter scale, of the diameter of the fundus vaginalis. The ovoid distal end thereof allows insertion into the vagina without the risk of unexpected lesions occurring. It provides the appropriate dimensions of the vaginal fornix (FIG. 3-a) up to a maximum of 13 centimeters.

### RELATED APPLICATIONS

This application is a U.S. national stage application under 35 U.S.C. 371 of International Application No. PCT/BR2013/000161 filed May 10, 2013 claiming priority of Brazil Application No. BR 102020132613 filed Jun. 1, 2012.

### TECHNICAL FIELD

The term colpometer is made up of the Greek prefix kolpos, meaning vagina, and the suffix meter, meaning to measure. The use of the present medical instrument called a colpometer (FIG. 2) is to measure the diameter of the vaginal fornix and thus facilitate the work of the health professional who requires this measurement for the application, for example, of an intravaginal device.

### DETAILED DESCRIPTION

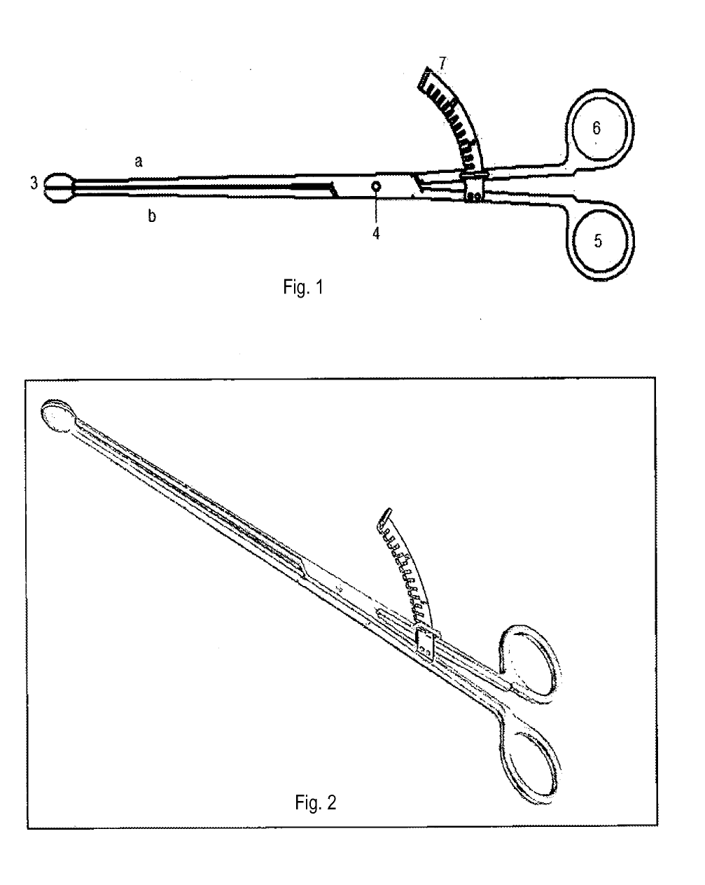

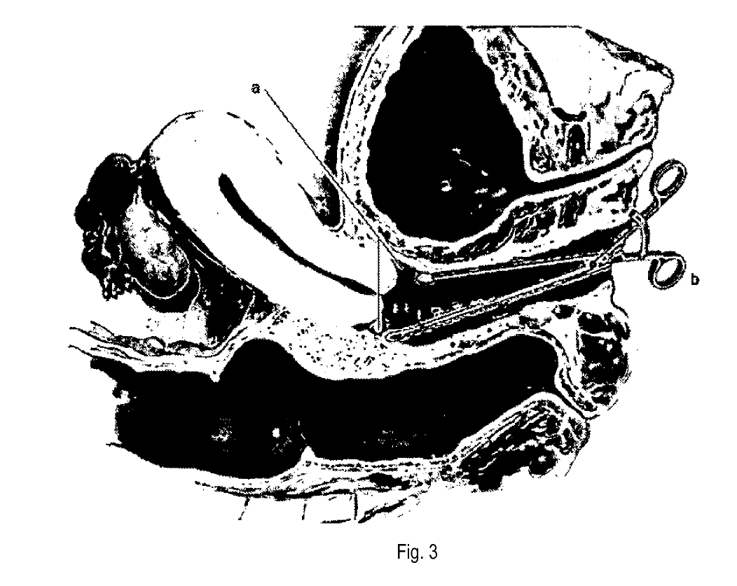

Its structure is similar to surgical forceps (FIGS. 1 and 2), characterized in that it has two arms (FIG. 1-a,b) with the center of rotation (FIG. 1-4) 149 mm from its distal end and 115 mm from the proximal end (handle) giving a total length of 264 mm. The distal end has an ellipsoidal format (FIG. 1-3) of a suitable shape for protecting the patient during its insertion into the vagina (FIG. 3-b). The handle, the proximal end, contains two circular loops (FIG. 1-5,6) for inserting the thumb in one and the ring finger in the other, like a pair of scissors. A curved arm is fixed to the handle or left proximal arm (FIG. 1-5), said curved arm (FIG. 1-7) containing a scale graduated in centimeters that is related to the opening of the handle as well as to the opening of the distal arms (FIG. 1-a,b). As the instrument is opened, the graduated scale gives the distance, in centimeters, that corresponds to the distance between the two ends of the distal arms (FIG. 1-a,b). If the distal end is spread to its maximum, the scale will show this distance equivalent to 13 centimeters.

FIG. 1—front view of the object—shows the instrument with its basic features mentioned above: the handle with the loops for use by the thumb and ring finger (FIG. 1-5,6); the graduated scale in centimeters (FIG. 1-7), which will supply the external distance of the spread of the distal arms (FIG. 1-a,b); it also shows the distal end of the forceps in ellipsoidal format when brought together and hemiellipsoidal when spread apart (FIG. 1-3); the center of rotation of the arms of the instrument (FIG. 1-4) allows, controlled by the fingers inserted in the handle, sliding and spreading of the end of the object, hence obtaining the required measurement. The end of the distal arms of the colpometer is of ovoid format and in this way allows its introduction in the vagina without risk of causing accidental injuries, since its ellipsoidal conformation is anatomical and protective.

It is an instrument made of steel AISI 420 that can be sterilized in an autoclave and reused whenever necessary. Another special feature of the instrument is that it can be made from polyethylene polymer for the purpose of producing a disposable measuring instrument.

1.-3. (canceled)

4. A colpometer for measuring the diameter of the vaginal fornix, comprising:
two shafts hinged at a center of rotation of the colpometer intermediate a distal arm at a distal end and a proximal arm at a proximal end of each shaft to allow concomitant opening of the distal arms whose distance between the external sides of one of the distal ends and the contralateral distal end is required to be determined,
a scale graduated in units of length on a curved arm located on the proximal arm of one of the two shafts,
a handle including a loop at the proximal end of each of the proximal arms of the two shafts for inserting a thumb and a finger,
wherein the distal arms of the two shafts are straight so as to close together, and
wherein the distal end of the distal arm of each of the two shafts is ovoid and smooth in an anatomical format.

5. The colpometer according to claim 4, wherein the distal ends of the distal arms of the two shafts have an ellipsoidal format when brought together and a hemiellipsoidal format when spread apart.

6. The colpometer according to claim 4, made of steel AISI 420.

7. The colpometer according to claim 4, made of polyethylene polymer.

8. The colpometer according to claim 4, wherein the scale is graduated in centimeters on the curved arm.

9. The colpometer according to claim 4, wherein the two loops have a diameter of two centimeters.

10. The colpometer according to claim 4, wherein the center of rotation is located 149 mm from the distal end and a 115 mm from the handle end.
